(19) United States
(12) Patent Application Publication
(10) Pub. No.: US 2020/0008706 A1
(43) Pub. Date: Jan. 9, 2020

(54) METHOD FOR PROVIDING HEALTH CARE INFORMATION BY USING CLOUD-BASED PORTABLE DEVICE FOR MEASURING BODY FAT AND DEVICE USING SAME

(71) Applicant: ONESOFTDIGM CO., LTD, Pohang-si (KR)

(72) Inventor: Dae Ho LEE, Pohang-si (KR)

(21) Appl. No.: 16/465,521

(22) PCT Filed: Nov. 30, 2017
(86) PCT No.: PCT/KR2017/013963

§ 371 Date: Jun. 25, 2019

(30) Foreign Application Priority Data
Dec. 2, 2016 (KR) 10-2016-0163237

(51) Int. Cl. A61B 5/053; A61B 5/00; G06F 3/01

(52) U.S. Cl. CPC A61B 5/0537; G06F 3/011; A61B 5/0022

(57) ABSTRACT

A method for providing health care information by using a cloud-based portable device for measuring body fat and a device using the same are disclosed. A health care information providing method is a method for allowing a cloud-based server to provide health care information, and comprises the steps of: collecting body fat measurement results from portable devices for measuring body fat of users; generating reference data for each group or each region on the basis of the collected body fat measurement results; and acquiring personal body fat measurement result information on the basis of the generated reference data.

### CLAIM FOR PRIORITY

This application claims priority to and the benefit of Korean Patent Application No. 10-2016-0163237, filed on Dec. 2, 2016, the disclosure of which is incorporated herein by reference in its entirety.

### TECHNICAL FIELD

The present invention relates to a method of providing healthcare information, and more particularly, to a method of providing healthcare information using a cloud-based portable device for measuring body fat and a device using the method.

### BACKGROUND ART

Body composition analysis is a method of quantitatively analyzing the composition of a human body and obtaining basic information for determining body status. The composition of a human body includes body water, protein, minerals, body composition, and the like.

Body fat indicates a person's ratio of fat to weight. Body fat may be classified as visceral fat and subcutaneous fat. Body fat varies much with individuals and may also varies with individuals' eating habits and amounts of exercise. In general, adult males have about 15 to 20% body fat, and adult females have about 20 to 25% body fat.

To measure body fat, bioelectrical impedance analysis (BIA) simply referred to as electrical resistance measurement is used most frequently.

BIA is a method of measuring body water by obtaining an impedance index when a small alternating current is applied to a human body on the basis of the principle that electric conductivity, that is, resistance, varies with the amount of water in a human body.

To obtain high precision and high reproducibility, a method of measuring impedance with multiple frequencies, a method of measuring impedance according to body parts, and the like are being used recently.

However, since each individual's eating habit and amount of exercise vary almost every day, a conventional body fat measurement technique shows a large deviation in each individual's body fat measurement results. Therefore, it is difficult to ensure precision and reproducibility in body fat measurement.

### DISCLOSURE

### Technical Problem

To solve the aforementioned problem, the present invention is directed to providing a method of providing healthcare information using a cloud-based portable device for measuring body fat (hereinafter, a healthcare information providing method) in which it is possible to collect body fat measurement results from the portable device for measuring users' body fat, generate group- and/or region-specific reference data on the basis of the collected individual body fat measurement results, and obtain highly precise individual body fat measurement result information on the basis of the generated reference data, and a device using the method.

The present invention is also directed to providing a method of providing healthcare information using a cloud-based portable device for measuring body fat in which it is possible to provide healthcare information including individual body fat measurement result information, individual activity information, etc. on the basis of group- and/or region-specific reference data generated by analyzing collected body fat measurement results, and a device using the method.

The present invention is also directed to providing a user terminal which acquires individual body fat measurement result information from a cloud-based server and a cloud-based portable device which is connected to the user terminal and measures the body composition of a user.

### Technical Solution

One aspect of the present invention provides a method of providing healthcare information from a cloud-based server, the method including collecting body fat measurement results from portable devices for measuring a user's body fat, generating group- or region-specific reference data on the basis of the collected body fat measurement results, and acquiring individual body fat measurement result information on the basis of the generated reference data.

The generating of the reference data may further include determining whether a preset group or region corresponds to the collected body fat measurement results and generating a group or region corresponding to the collected body fat measurement results when any preset group or region does not correspond to the collected body fat measurement results.

The generating of the reference data may further include, after the generating of the group or region, determining whether the collected body fat measurement results are within a preset average range of the group or region, generating an exceptional group of the group or region when the collected body fat measurement results are not within the preset average range, and applying a weight preset according to a distribution of pre-collected body fat measurement results belonging to the exceptional group and/or a ratio of the pre-collected body fat measurement results to the body fat measurement results of the group or region.

The generating of the reference data may further include, after the applying of the weight, generating reference data on the basis of collected information stored in connection with the group or region and stored collected information to which the weight has been applied.

Another aspect of the present invention provides a user terminal which is connected to a cloud-based server via a network and has a mobile application for outputting individual body fat measurement results, the user terminal including a memory configured to store a program and a processor configured to be connected to the memory and execute the program. The program includes a body fat analysis module, a step count recognition module, and a health data collection module. Due to the program, the processor acquires body fat measurement results from a portable device for measuring a user's body fat, transmits the acquired body fat measurement results to the cloud-based server, and acquires individual body fat measurement results based on reference data, which is generated on the basis of body fat measurement results collected according to groups or regions, or information on the individual body fat measurement results from the cloud-based server.

The reference data may be generated on the basis of an average of first collected information within an average range preset according to the groups or regions and second collected information which is not within the average range and to which a weight has been applied.

Still another aspect of the present invention provides a portable device which is connected to a user terminal via a wireless network and measures a user's body fat, the portable device including a first pair of terminals with which a thumb and one of other fingers of the user's left hand each come in contact, a second pair of terminals with which a thumb and one of other fingers of the user's right hand each come in contact, at least one signal detection circuit configured to be connected to the first pair of terminals and the second pair of terminals and measure a signal related to the user's body composition or healthcare, a controller configured to be connected to the signal detection circuit, a sensor configured to be connected to the controller and detect vibration or acceleration, and a communication unit configured to be connected to the controller and connected to the user terminal via a communication network. The controller transmits the signal or information corresponding to the signal to a cloud-based server through the user terminal. The cloud-based server classifies collected body fat measurement results by preset group or region, acquires individual body fat measurement results on the basis of reference data generated on the basis of a group- or region-specific average, and provides the individual body fat measurement results to the user terminal.

The reference data may be generated on the basis of an average of first collected information within an average range preset according to groups or regions and second collected information which is not within the average range and to which a weight has been applied.

### DESCRIPTION OF DRAWINGS

FIG. 1 is a schematic diagram showing a configuration of a healthcare system according to an embodiment of the present invention.

FIG. 2 illustrates main operating principles of the healthcare system of FIG. 1.

FIG. 3 is a perspective view of a portable device that can be employed in the healthcare system of FIG. 1.

FIG. 4 is a top-down view of the portable device of FIG. 3.

FIG. 5 is a front view of the portable device of FIG. 4.

FIG. 6 shows the portable device of FIG. 3 in use.

FIG. 7 is a schematic block diagram showing a configuration of the portable device of FIG. 3.

FIG. 8 is a schematic block diagram showing a configuration of a user terminal that can be employed in the healthcare system of FIG. 1 according to an embodiment of the present invention.

FIG. 9 is a graph illustrating the position of a representative value for adaptive threshold algorithm (ATA) application to activity measurement that can be employed in a healthcare system of FIG. 1.

FIG. 10 is a graph illustrating a step count detection algorithm in the activity measurement of FIG. 9.

FIG. 11 is a block diagram illustrating a configuration of a user terminal that can be employed in the healthcare system of FIG. 1 according to another embodiment.

FIG. 12 is a sequence diagram illustrating operating principles of a user terminal that can be employed in the healthcare system of FIG. 1 according to another embodiment.

FIG. 13 is a flowchart of a method of providing healthcare information according to another embodiment of the present invention.

FIG. 14 illustrates operating principles of the healthcare information providing method of FIG. 13.

FIGS. 15A to 15D illustrate operating principles of the healthcare information providing method of FIG. 13 according to other embodiments.

FIG. 16 illustrates operating principles of the healthcare information providing method of FIG. 13 according to another embodiment.

FIG. 17 illustrates operating principles that can be employed in the healthcare information providing method of FIG. 13 according to another embodiment.

FIG. 18 illustrates operating principles of FIG. 17 according to another embodiment.

### MODES OF THE INVENTION

As the present invention allows a variety of modifications and have various embodiments, particular embodiments will be illustrated in the drawings and described in detail. However, this is not intended to limit the present invention to particular modes of practice, and it is to be appreciated that all modifications, equivalents, and substitutes included in the spirit and technical scope of the present invention are encompassed in the present invention. Throughout the drawings, like reference numerals are used for like elements.

Although the terms “first,” “second,” “A,” “B,” etc. may be used to describe various elements, these elements should not be limited by these terms. The terms are only used to distinguish one element from other elements. For example, without departing from the scope of the present invention, a first element may be termed a second element, and similarly, a second element may be termed a first element. The term “and/or” includes any and all combinations of a plurality of listed relevant items.

It will be understood that when an element is referred to as being “connected” or “coupled” to another element, it can be directly connected or coupled to the other element, or intervening elements may be present. In contrast, it will be understood that when an element is referred to as being “directly connected” or “directly coupled” to another element, there are no intervening elements present.

The terminology used herein is for the purpose of describing particular embodiments only and is not intended to limit the invention. The singular forms include the plural forms as well, unless the context clearly indicates otherwise. It will be understood that the terms “include,” “have,” etc. specify the presence of stated features, numbers, steps, operations, elements, components, or combinations thereof and do not preclude the presence or addition of one or more other features, numbers, steps, operations, elements, components, or combinations thereof.

As used herein, when a subscript or superscript of any character has another subscript or superscript, the other subscript or superscript may be displayed in the same fashion as the subscript or superscript for the convenience of expression unless there is no misunderstanding.

Unless otherwise defined, all terms, including technical and scientific terms, used herein have the same meaning as commonly understood by those of ordinary skill in the art to which the present invention pertains. Terms, such as those defined in commonly used dictionaries, should be interpreted as having a meaning that is consistent with their meaning in the context of the relevant art and will not be interpreted in an idealized or overly formal sense unless expressly so defined herein.

Among terms used in this embodiment, some major terms are defined as follows.

Healthcare may refer to an overall industry related to users' health. Healthcare may include not only medical services, such as conventional treatment, but also services related to dietary supplements, food, cosmetics, etc. for disease prevention and management and health.

Healthcare information may refer to overall information related to users' health. Healthcare information may include at least one piece of information selected from body composition information, medical information, bio-related information, activity information, and the like.

Hereinafter, exemplary embodiments of the present invention will be described in detail with reference to the accompanying drawings.

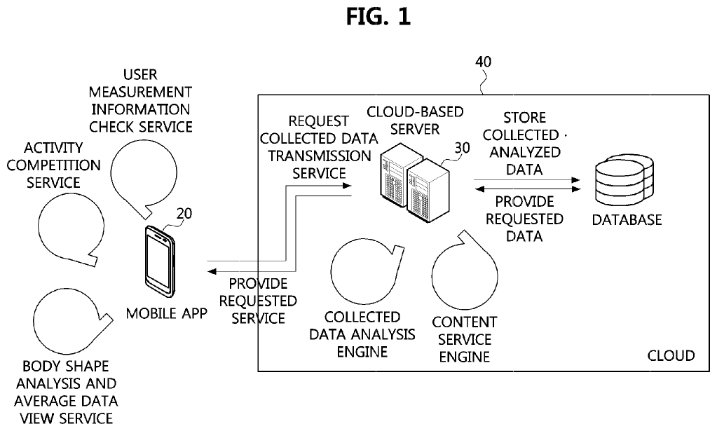

FIG. 1 is a schematic diagram showing a configuration of a healthcare system according to an embodiment of the present invention.

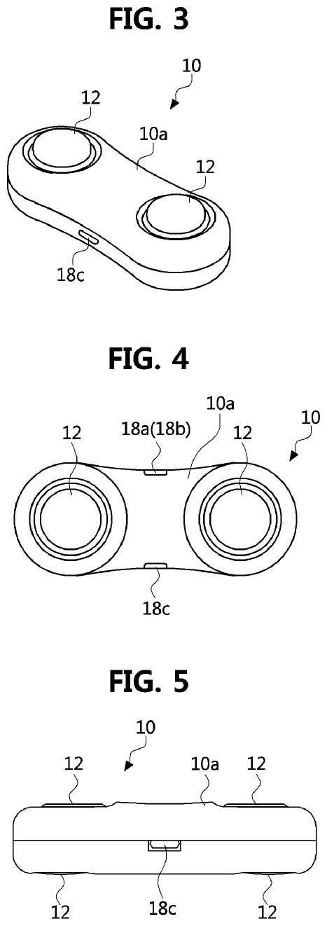

Referring to FIG. 1, the healthcare system according to this embodiment may include a portable device (see 10 in FIG. 3), a user terminal 20, and a cloud-based server 30. A mobile application (app.) may be installed on the user terminal.

The user terminal 20 may acquire body composition information and/or activity information through the portable device, transmit the collected data to the cloud-based server 30 or request a healthcare service from the cloud-based server 30, and receive the service from the cloud-based server 30.

The mobile app. which is installed on the user terminal 20 and interoperates with the portable device or the cloud-based server 30 may include a measurement device (or portable device) interoperation module, a user management and login module, a collected data transmission module, an other device interoperation and setting module, and the like. The user terminal 20 may provide a user measurement information check service, an inter-user activity competition service, a body shape analysis and region-based measurement data (or average data) view service, etc. to a user on the basis of data or information from the portable device and/or a service or information provided by the cloud-based server 30.

The cloud-based server 30 may be connected to a database server for managing a database, store collected or analyzed data, and receive stored data by requesting the data. The cloud-based server and the database (or a database server) may be referred to as a cloud 40. The cloud-based server 30 may include a cloud-based service server, a collected data analysis and storage module (or a collected data analysis engine), a content service engine, etc. to provide healthcare information.

According to this embodiment, in the healthcare system, the cloud-based server may basically generate group- and/or region-specific reference data on the basis of data or information (e.g., body composition information and activity information) measured by the portable device and provide reliable individual healthcare measurement results (healthcare information) on the basis of the generated reference data.

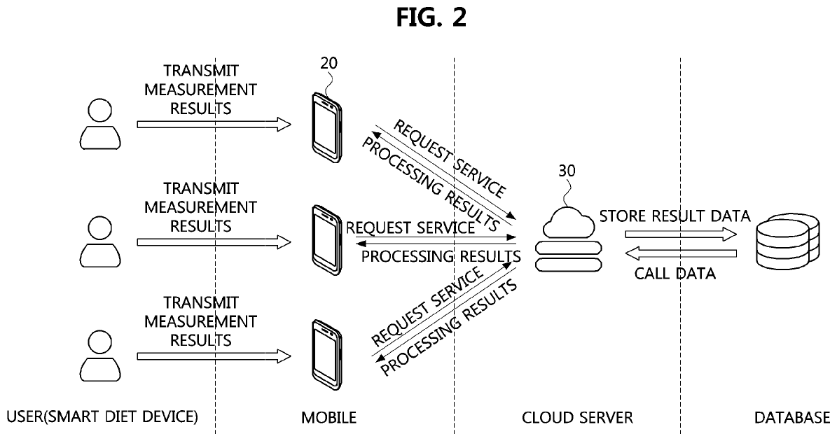

FIG. 2 illustrates main operating principles of the healthcare system of FIG. 1.

Referring to FIG. 2, in the healthcare system of this embodiment, the portable device measures a user's body composition, amount of activity, etc. and transmits measurement results to the user terminal 20. The portable device may be referred to as a smart diet device, and a user terminal 20 of each user may be a mobile device.

A plurality of user terminals 20 may transmit data or information measured by portable devices to the cloud-based server (in brief, cloud server) 30 and request a service based on the data or information. The service may include healthcare information, such as body fat measurement results and activity analysis results. Also, each user terminal 20 may receive or acquire processing results from the cloud server 30 in response to the service request. The above-described user terminals 20 may have the mobile app. installed thereon. In this case, the mobile app. may have an interoperation module for interoperating with a portable device or an open application programming interface (API) for the expandability of an interoperation module.

The cloud server 30 may store collected data from the user terminals 20 and result data obtained by analyzing the collected data in the database, call data as necessary, and transmit processing results to the user terminals 20.

Also, the cloud server 30 may be built as a service providing server using a commercial cloud service in order to efficiently manage and run infrastructure and reduce maintenance costs. The commercial cloud service may include AkWS, MS Azure, and the like.

According to this embodiment, the cloud-based server 30 may generate reference data on the basis of data collected from the plurality of user terminals 20, compare individually measured body fat measurement results or activity measurement results with the reference data, and provide healthcare information including individual body fat measurement results, individual activity measurement results, etc. based on the reference data to the user terminals.

According to this embodiment, a conventional service structure in which data is stored in a mobile device is improved, and it is possible to collect and store information collected from portable body composition measuring devices (portable devices) through a cloud-based server and provide various wellness services or healthcare information to application users using the collected information.

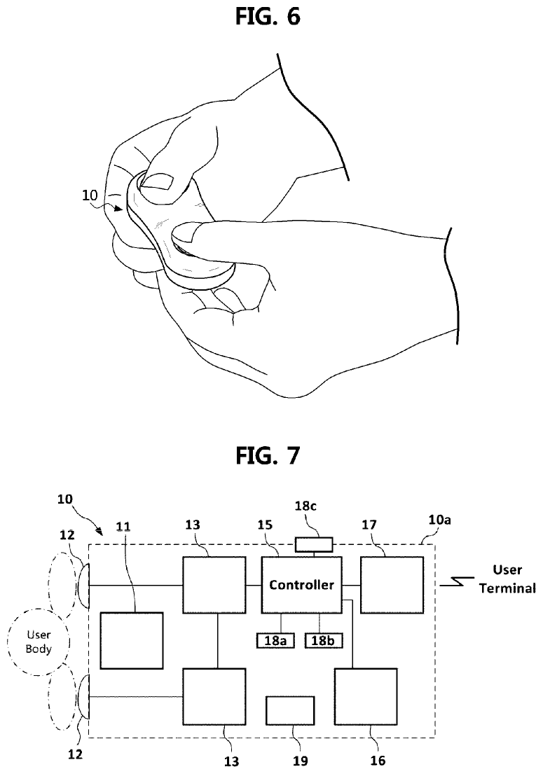

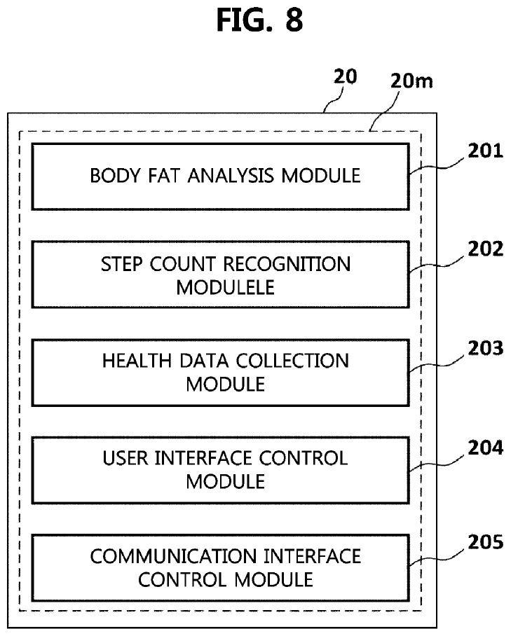

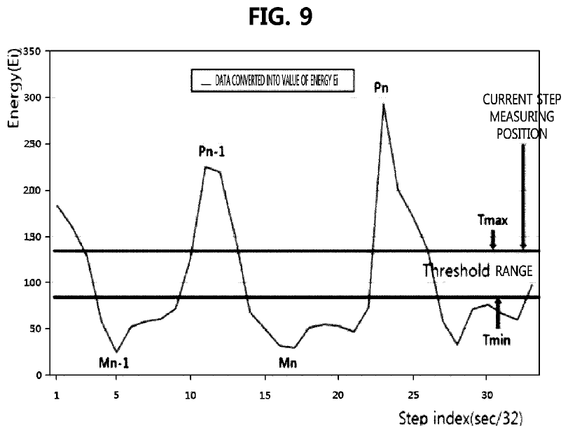

FIG. 3 is a perspective view of a portable device that can be employed in the healthcare system of FIG. 1. FIG. 4 is a top-down view of the portable device of FIG. 3. FIG. 5 is a front view of the portable device of FIG. 4. FIG. 6 shows the portable device of FIG. 3 in use.

Referring to FIG. 3, the portable device 10 of this embodiment may have a peanut shape, a length of about 6 to 7 cm in a direction of the longest length (a longitudinal direction), a length of about 3 cm in a first direction crossing at right angles with the longitudinal direction (a transverse direction), and a length of about 1.5 cm in a second direction crossing at right angles with the longitudinal direction and the transverse direction (a thickness direction). The longitudinal direction may extend to the left and right of the ground of FIG. 4 or 5, the transverse direction may extend upward and downward from the ground of FIG. 4. The thickness direction may extend upward and downward from the ground of FIG. 5.

The portable device 10 may include a peanut-shaped case 10a, two terminals 12 each exposed in an upper surface and a lower surface of the case 10ain the thickness direction, a reset button 18a exposed in one side surface of the case 10ain the thickness direction, and a connector 18c exposed in the other side surface which is the opposite side surface of the one side surface of the case 10a. The reset button 18a may be arranged integrally or in combination with a light-emitting structure 18b, such as a light-emitting diode (LED).

The case 12a may be formed of a plastic material, a natural polymer material, or the like. The plastic may refer to a polymer compound or a synthetic polymer material that can be shaped by applying heat or pressure. Two terminals exposed in the upper surface and the lower surface on one side of the case 12a may be referred to as a first pair of terminals, and two terminals exposed in the upper surface and the lower surface on the other side of the case 12a may be referred to as a second pair of terminals.

According to this embodiment, as shown in FIG. 6, a user may grab the first pair of terminals of the portable device 10 with the thumb and one of the other fingers of his or her left hand, grab the second pair of terminals with the thumb and one of the other fingers of his or her right hand, and measure his or her body composition. Also, the user may carry the small-sized portable device and measure his or her amount of activity while moving.

The portable device employed in the healthcare system of this embodiment may be referred to as a portable body composition/activity measuring device. Body composition measurement results and/or activity measurement results may be transmitted from the portable device to a user terminal or the cloud-based server periodically at preset time intervals, intermittently at preset times, or automatically. In other words, the cloud-based server may collect users' body composition data and activity data measured by portable devices using user terminals (which include mobile smart devices), store the collected data in the database, generate average body composition or the average amount of activity based on user regions and/or user groups on the basis of the collected data, and provide an inter-user competition service or reliable individual body composition measurement results on the basis of the average body composition or the average amount of activity.

Also, according to the configuration of the portable device 10 described above, a user terminal or the cloud-based server may include an activity check module, a social service providing module, a healthcare information guide module, a module for providing a measurement value statistics service, and the like. Accordingly, a user may use various kinds of information and data related to healthcare through a service app., a social function, etc. that his or her user terminal has.

Further, in a situation in which the number of extremely obese twenties and thirties is rapidly increasing due to a socio-culturally changing living environment such as rapid proliferation of fast food, an increase in the use of owner-driven cars, and desk work in office, it is possible to provide an environment for efficiently managing a user's health with high reliability.

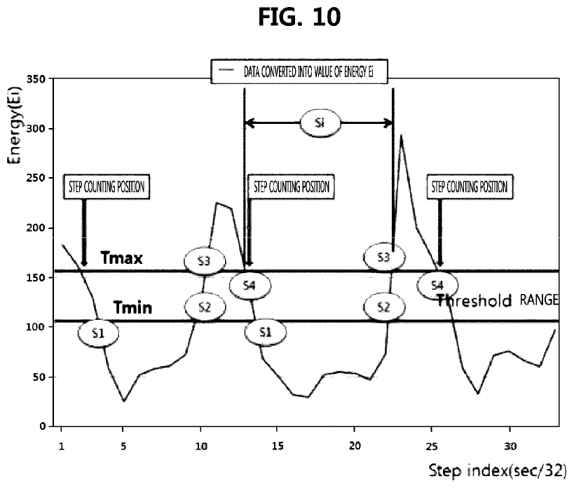

FIG. 7 is a schematic block diagram showing a configuration of the portable device of FIG. 3.

Referring to FIG. 7, the portable device 10 according to this embodiment may include the case 10a, an anti-static circuit 11, terminals 12, a signal detection circuit 13, a controller 15, a sensor 16, a communication unit 17, a reset button 18a, a light-emitting structure 18b, a connector 18c, and a power supply 19. The anti-static circuit 11, the reset button 18a, the light-emitting structure 18b, the connector 18c, or a combination thereof may be omitted according to implementation.

The anti-static circuit 11 may include a resistor which is serially connected between the terminals 12 and the signal detection circuit 13 and a capacitor between the ground and a node or a contact point between the resistor and the signal detection circuit 13.

The terminals 12 may include the first pair of terminals and the second pair of terminals. The signal detection circuit 13 may measure a bioelectric resistance flowing in the terminals 12. The bioelectric resistance refers to a resistance value obtained when a small alternating current (e.g., 800 μA) flows to a human body on the basis of the principle that the conductance of a human body composed of moisture, which is highly electrically conductive, varies according to the amount of moisture.

Bioelectrical impedance analysis (BIA) is based on the electrical characteristic that a human body is composed of tissue with high conductivity (conductor) and tissue with low conductivity (insulator). BIA makes it possible to measure a resistance against electricity flowing through moisture, which is highly conductive, by applying a small current signal to a human body. Since such a resistance has a certain correlation with body composition, such as body water, fat, and muscle, which show different electrical characteristics according to their amounts of moisture, it is possible to measure body composition on the basis of the resistance.

The signal detection circuit 13 may measure body composition according to the BIA principle. The signal detection circuit 13 may include an equivalent circuit of a circuit obtained by serially connecting a first equivalent resistance corresponding to an intracellular (cytoplasm) fluid resistance to a parallel circuit of an equivalent capacitance and an equivalent resistance representing capacitance and resistance of a cell membrane and connecting a second equivalent resistance corresponding to an extracellular fluid resistance in parallel to the serial circuit of the parallel circuit and the first equivalent resistance.

The controller 15 may sense a signal or data from the signal detection circuit 13 and output a signal, data, or information corresponding to the sensed signal or data. The signal or data may include a signal or data related to body fat. The signal, data, or information output from the controller 15 may be transferred to a user terminal through the communication unit 17.

The sensor 16 may include an accelerometer. The accelerometer may be implemented as a capacitive sensing type which generates an electrical output signal from a displacement caused by an acceleration input. The sensor 16 may be implemented as an analog signal processing circuit, such as a sine wave oscillator circuit, a charge integration circuit, an amplification circuit, a demodulation circuit, a filter circuit, and the like. In this case, the sensor 16 may detect a capacitance variation caused by a displacement between a mass and a sensing electrode according to an external acceleration. In other words, when an external acceleration is applied, a capacitance between the sensing electrode and the mass is varied by the displacement. Assuming that the capacitance variation is in a linear relationship with a displacement of the gap in a small displacement section, it is possible to estimate an acceleration input.

When the accelerometer is used, it is possible to sense vibrations which are made every time a user takes a step by processing a signal through x, y, and z, 3-axis sensing and calculate the user's number of steps through the vibrations. The number of steps may be transferred to the user through an activity data interface.

The communication unit 17 may include a short-range wireless communication module such as a Bluetooth module. The communication unit 17 may be connected to the controller 15 and connect the controller 15 and the user terminal via a wireless network.

The reset button 18a may be connected to the controller 15 and receive or generate a user input for initializing an operating mode of the portable device 10. The reset button 18a may be exposed on one side surface of the case 10a.

The light-emitting structure 18b may include an LED or the like. The light-emitting structure 18b may be integrated or combined with the reset button 18a and implemented to emit light from the surface or vicinity of the reset button 18a.

The connector 18c may provide a connection point of a physical interface which connects the portable device 10 to an external device through a conductive cable and enables exchange of a signal, data, etc. between the portable device 10 and the external device or enables the external device to manage the portable device 10. As the connector 18c, a universal serial bus (USB) connector conforming to USB, which is a serial bus standard, may be used.

The power supply 19 may supply power to electronic parts in the portable device 10. The power supply 19 may include a voltage source, a current source, or both of them. The power supply 19 may include a battery, a wireless charging circuit, a commercial power adapter, or a combination structure thereof.

When the power supply 19 includes a battery, the battery power may be rapidly consumed by operation of the sensor 16. To flexibly handle battery consumption, the portable device 10 of this embodiment may include a removable battery structure as at least one component of the power supply 19.

When the portable device 10 of this embodiment is used, it is possible to visualize and show accurate measurement result values of healthcare information, such as body fat and the amount of activity, in conjunction with a user terminal, such as a smart phone, and also possible to provide a service or a service development environment in which the measurement result values are used.

The service may involve providing personal data, such as body fat, the amount of muscle, daily calorie burn, and the amount of activity, comparing the personal data with a regional or group average or reference data and providing accurate customized measurement result values with high reliability, or providing various wellness services and convergence services based on the personal data and/or the customized measurement result values.

In terms of data view manner, the customized measurement result values do not merely show measurement results and a history, unlike a conventional wearable app. Rather, the customized measurement result values may motivate a user who has a below-average amount of activity or an above-average amount of body fat to actively manage his or her body by showing an average value of the corresponding region (e.g., a country) or a relevant group (e.g., an age group or a human race group).

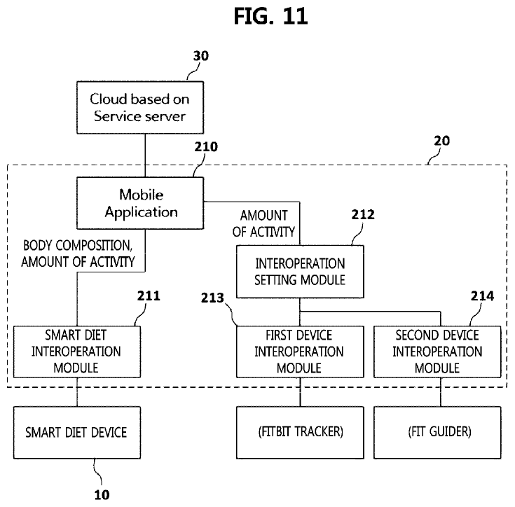

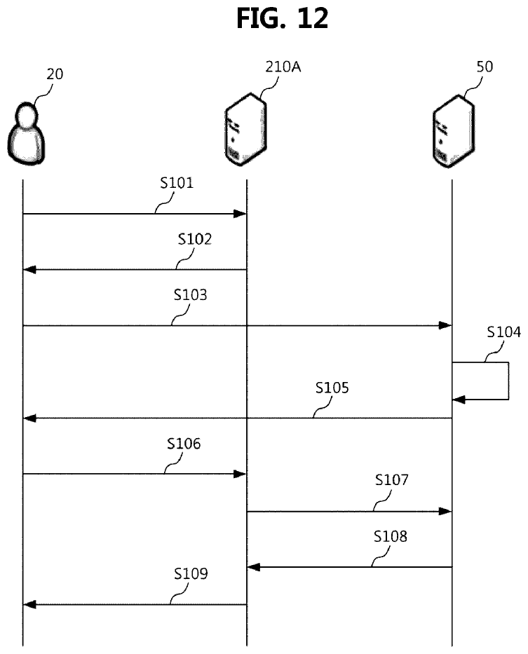

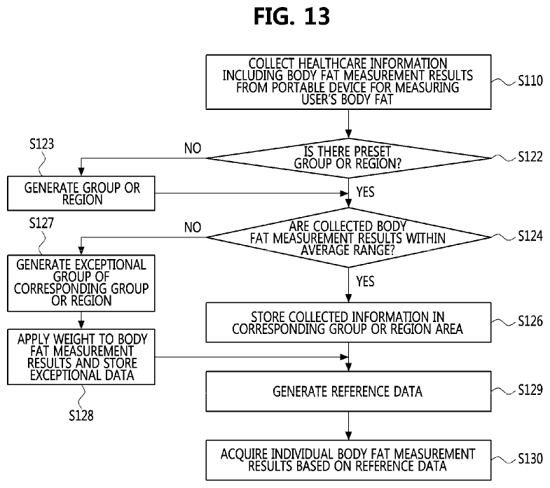

FIG. 8 is a schematic block diagram showing a configuration of a user terminal that can be employed in the healthcare system of FIG. 1 according to an embodiment of the present invention. FIG. 9 is a graph illustrating the position of a representative value for adaptive threshold algorithm (ATA) application to activity measurement that can be employed in a healthcare system of FIG. 1. FIG. 10 is a graph illustrating a step count detection algorithm in the activity measurement of FIG. 9.

Referring to FIG. 8, the user terminal 20 of this embodiment may include a body fat analysis module 201, a step count recognition module 202, a health data collection module 203, a user interface (UI) control module 204, and a communication interface control module 205. These modules 201 to 205 may be stored or installed in a memory system or a memory 20m of the user terminal 20 in the form of software modules. According to implementation, the modules 201 to 205 can also be installed in a processor which is connected to the memory and executes a program or a software module stored in the memory.

The body fat analysis module 201 may analyze a user's body fat on the basis of body composition measurement information acquired from a portable device. The body fat analysis may be performed using BIA. The body fat analysis module 201 may be implemented in the form of a program, a software module, or a mobile application.

The step count recognition module 202 may include the sensor included in the portable device 10 which has been described above with reference to FIG. 7 and an at least partial function unit or constituent unit of a controller for analyzing the user's activity on the basis of an output signal of the sensor. In this case, the at least partial function unit of the controller may correspond to an activity analysis module.

In other words, it is possible to calculate the number of steps by analyzing data of the acceleration sensor. In this embodiment, an ATA method may be used. When the ATA method is used, it is possible to increase a step count recognition rate by adjusting a maximum threshold value Tmax and a minimum threshold value Tmin.

In the ATA used in step count recognition, the relationship between the maximum threshold value and the minimum threshold value is as shown in [Equation 1] below.

Tmax=Tmin+(\sqrt{|K−Tmin|}×C₂) [Equation 1]

In [Equation 1], K is a condition for updating a threshold range. When first maximum peak data of a previous step is smaller than second maximum peak data of another previous step immediately before the previous step, K is set to the first peak data value. When the first maximum peak data is greater than or equal to the second maximum peak data, K is set to the second maximum peak data value. C2 is a value determined by experiments.

The portable device and/or the user terminal of this embodiment may set a position of a representative value for ATA application to the sensor or the accelerometer installed in the portable device. As shown as an example in FIG. 9, the maximum threshold value Tmax and the minimum threshold value Tmin may be adjusted according to a current step measuring position. This is intended to adjust signals which are output from the sensor in different forms when the portable device is attached to the user's upper pocket, trouser pocket, arm, etc. or put into a purse or a bag.

In FIG. 9, Pn indicates maximum peak data of a previous step, Pn−1 indicates maximum peak data of a previous step of Pn, Mn indicates minimum peak data of the previous step, and Mn−1 indicates minimum peak data of the previous step of Mn.

An application example of a step count detection algorithm to which the above-described ATA is applied is shown in FIG. 10. FIG. 10 shows data of three steps. In FIG. 10, the number of steps may be measured as follows. Output data of the acceleration sensor may be converted into an energy value Ei and input. While a value S1 smaller than Tmin is input, the input is continuously received, and at a point or time S2 at which the energy value becomes greater than Tmin, Tmin is updated. While the energy value is greater than Tmax of the threshold range (S3), Tmax is continuously updated, and when the energy value becomes smaller than Tmax (S4), a step is counted.

In [Table 1] below, the accuracy of a step number is compared between the step count recognition method (using the ATA) of this embodiment and a step count recognition method (using a heuristic algorithm (HA)) of a comparative example. The portable device of this embodiment is disposed on the user's arm or waist.

TABLE 1

| | | | Detected | Detected |
|---|---|---|---|---|
| Number of | | Number of | number | number |
| times | Classification | steps | (HA) | (ATA) |
| First time | Arm | 273 | 265(97.1) | 263(96.3) |
| | Waist | | 255(93.4) | 266(97.4) |
| Second time | Arm | 275 | 265(96.8) | 274(99.3) |
| | Waist | | 267(97.0) | 273(99.3) |
| Third time | Arm | 282 | 265(93.8) | 275(97.5) |
| | Waist | | 261(92.5) | 277(98.2) |
| Fourth time | Arm | 280 | 266(95.0) | 277(98.9) |
| | Waist | | 268(95.7) | 269(96.1) |
| Fifth time | Arm | 273 | 252(92.3) | 260(95.2) |
| | Waist | | 258(94.5) | 265(97.1) |

As shown in [Table 1], the recognition rate of the method of this embodiment is improved by about 3%.

Referring back to FIG. 8, the health data collection module 203 may be implemented by a Tomcat-based Web Application Server (WAS) or the like. As a combination of a web server and a web container, the WAS may refer to a server which performs various roles by implementing various functions in a container. The health data collection module 203 may include the definition of a communication protocol between an application (a mobile app., a service app., etc.) and a server and store collected data in the database.

A WAS-App communication protocol is defined as shown in [Table 2] below, and in this case, data values of the database may be displayed as shown in [Table 3].

TABLE 2

| Name | Type | Description |
|---|---|---|
| get_uid | string | user information |
| get_insert_dt | date | storage date and time |
| get_avrweight | float | weight |
| get_battery | int | remaining battery capacity |
| get_bodyfat | float | body fat ratio |
| get_fatsscore | float | amount of body fat |
| get_gender | string | sex |
| get_height | float | height |
| get_lastheight | float | last height |
| get_lastbodyfat | float | last body fat ratio |
| get_lastweight | float | last weight |
| get_musclescore | float | amount of muscle |
| get_bluetooth | int | identification number of machine |

TABLE 3

| | | | | | BODY | FATS | |
|---|---|---|---|---|---|---|---|
| uid | INSERT_DT | AVRWEIGHT | BATTERY | BODY_FAT | TYPE | SCORE | GENDER |
| 171425 | 2015-11-30 | 46.818 | 100 | 26 | 6 | −100 | 2 |
| | 17:42:41.483 | | | | | | |
| 171476 | 2015-11-30 | 46.818 | 100 | 23.7 | 0 | 30 | 1 |
| | 20:49:47.477 | | | | | | |
| 171474 | 2015-12-01 | 46.818 | 100 | 19.3 | 0 | 4 | 1 |
| | 20:40:42.567 | | | | | | |
| 171475 | 2015-12-01 | 46.818 | 100 | 18.8 | 6 | −27 | 2 |
| | 20:43:03.010 | | | | | | |
| 171410 | 2015-12-02 | 63.579998 | 100 | 21.9 | 0 | 0 | 1 |
| | 15:48:52.553 | | | | | | |
| 171410 | 2015-12-02 | 63.579998 | 100 | 25.8 | 0 | 17 | 1 |
| | 15:56:39.523 | | | | | | |
| 171488 | 2015-12-02 | 70.4902 | 100 | 24.9 | 0 | 0 | 1 |
| | 16:27:54.883 | | | | | | |
| 171478 | 2015-12-02 | 62.092796 | 77.8 | 25.3 | 0 | 79 | 1 |
| | 21:29:21.757 | | | | | | |
| 171478 | 2015-12-02 | 62.092796 | 76.4 | 32.9 | 0 | 133 | 1 |
| | 21:30:09.560 | | | | | | |
| 171410 | 2015-12-03 | 63.579998 | 100 | 27.7 | 0 | 43 | 1 |
| | 14:12:10.620 | | | | | | |
| 171400 | 2015-12-07 | 0 | 100 | 20.3 | 0 | −100 | 1 |
| | 14:38:38.797 | | | | | | |
| 171410 | 2015-12-07 | 63.579998 | 100 | 27.7 | 0 | −100 | 1 |
| | 14:40:00.240 | | | | | | |
| 171410 | 2015-12-07 | 63.579998 | 100 | 27.7 | 0 | −100 | 1 |
| | 14:40:30.210 | | | | | | |
| 171425 | 2015-11-30 | 46.818 | 100 | 26 | 6 | −100 | 2 |
| | 17:42:41.483 | | | | | | |
| 171476 | 2015-11-30 | 46.818 | 100 | 23.7 | 0 | 30 | 1 |
| | 20:49:47.477 | | | | | | |
| 171474 | 2015-12-01 | 46.818 | 100 | 19.3 | 0 | 4 | 1 |
| | 20:40:42.567 | | | | | | |
| 171475 | 2015-12-01 | 46.818 | 100 | 18.8 | 6 | −27 | 2 |
| | 20:43:03.010 | | | | | | |
| 171410 | 2015-12-02 | 63.579998 | 100 | 21.9 | 0 | 0 | 1 |
| | 15:48:52.553 | | | | | | |
| 171410 | 2015-12-02 | 63.579998 | 100 | 25.8 | 0 | 17 | 1 |
| | 15:56:39.523 | | | | | | |
| 171488 | 2015-12-02 | 70.4902 | 100 | 24.9 | 0 | 0 | 1 |
| | 16:27:54.883 | | | | | | |
| 171478 | 2015-12-02 | 62.092796 | 77.8 | 25.3 | 0 | 79 | 1 |
| | 21:29:21.757 | | | | | | |
| 171478 | 2015-12-02 | 62.092796 | 76.4 | 32.9 | 0 | 133 | 1 |
| | 21:30:09.560 | | | | | | |
| 171410 | 2015-12-03 | 63.579998 | 100 | 27.7 | 0 | 43 | 1 |
| | 14:12:10.620 | | | | | | |
| 171400 | 2015-12-07 | 0 | 100 | 20.3 | 0 | −100 | 1 |
| | 14:38:38.797 | | | | | | |
| 171410 | 2015-12-07 | 63.579998 | 100 | 27.7 | 0 | −100 | 1 |
| | 14:40:00.240 | | | | | | |
| 171410 | 2015-12-07 | 63.579998 | 100 | 27.7 | 0 | −100 | 1 |
| | 14:40:30.210 | | | | | | |

| | | LAST | LAST | LAST | MUSCLE | USERINFO | USERINFO |
|---|---|---|---|---|---|---|---|
| uid | HEIGHT | HEIGHT | PERFAT | WEIGHT | SCORE | SORTITEM | SORTORD |
| 171425 | 153 | 153 | 26 | 49 | −100 | 0 | 0 |
| 171476 | 171 | 171 | 23.7 | 70 | 57 | 0 | 0 |
| 171474 | 175 | 175 | 19.3 | 69 | 64 | 0 | 0 |
| 171475 | 161 | 161 | 18.8 | 49 | 10 | 0 | 0 |
| 171410 | 170 | 170 | 21.9 | 75 | 0 | 0 | 0 |
| 171410 | 170 | 170 | 25.8 | 75 | −5 | 0 | 0 |
| 171488 | 179 | 179 | 24.9 | 80 | 0 | 0 | 0 |
| 171478 | 180 | 180 | 25.3 | 92 | 1 | 0 | 0 |
| 171478 | 180 | 180 | 32.9 | 92 | −9 | 0 | 0 |
| 171410 | 176 | 176 | 27.7 | 85 | 4 | 0 | 0 |
| 171400 | 173 | 173 | 20.3 | 76 | −100 | 0 | 0 |
| 171410 | 162.4 | 176 | 27.7 | 85 | −100 | 0 | 0 |
| 171410 | 162.4 | 176 | 27.7 | 85 | −100 | 0 | 0 |
| 171425 | 153 | 153 | 26 | 49 | −100 | 0 | 0 |
| 171476 | 171 | 171 | 23.7 | 70 | 57 | 0 | 0 |
| 171474 | 175 | 175 | 19.3 | 69 | 64 | 0 | 0 |
| 171475 | 161 | 161 | 18.8 | 49 | 10 | 0 | 0 |
| 171410 | 170 | 170 | 21.9 | 75 | 0 | 0 | 0 |
| 171410 | 170 | 170 | 25.8 | 75 | −5 | 0 | 0 |
| 171488 | 179 | 179 | 24.9 | 80 | 0 | 0 | 0 |
| 171478 | 180 | 180 | 25.3 | 92 | 1 | 0 | 0 |
| 171478 | 180 | 180 | 32.9 | 92 | −9 | 0 | 0 |
| 171410 | 176 | 176 | 27.7 | 85 | 4 | 0 | 0 |
| 171400 | 173 | 173 | 20.3 | 76 | −100 | 0 | 0 |
| 171410 | 162.4 | 176 | 27.7 | 85 | −100 | 0 | 0 |
| 171410 | 162.4 | 176 | 27.7 | 85 | −100 | 0 | 0 |

The UI control module 204 may provide the functions of user registration, editing, management, etc. regarding healthcare information through a body composition measurement screen, the output of body composition measurement data, a body composition measurement result screen, an activity measurement screen, the output of activity measurement data, an activity measurement result screen, and the like. Also, the UI control module 204 may provide accumulated, weekly, and monthly graphs of measured data and provide the function of showing the graphs.

The communication interface control module 205 may have a device-app. interface for transmitting body composition measurement results or acceleration sensing data. Also, the communication interface control module 205 may transmit information on the number of steps through the device-app. interface. Further, the communication interface control module 205 may transmit information related to noise data removal, data of remaining battery capacity, and the like. The communication interface control module may include a data transmission interface which processes a host request or command (CMD) of Table 2 or 4 according to a device response command having a predetermined value and format.

According to this embodiment, a measurement environment for measuring body composition is not limited, and a portable device for measuring body composition can be easily carried. Therefore, a user can immediately measure his or her body composition or amount of activity anytime and anywhere as the user wants, and it is possible to frequently provide or acquire healthcare information through a community such as a social network service (SNS).

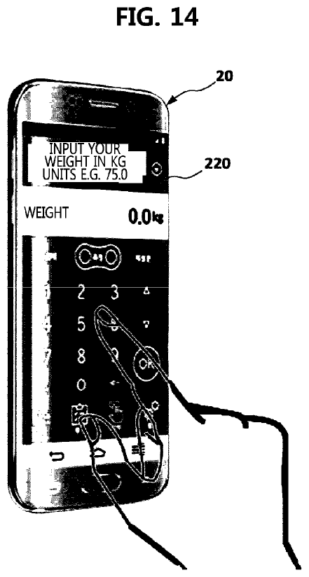

FIG. 11 is a block diagram illustrating a configuration of a user terminal that can be employed in the healthcare system of FIG. 1 according to another embodiment.

Referring to FIG. 11, a user terminal 20 according to this embodiment may include a mobile app. 210, a smart diet interoperation module 211, an interoperation setting module 212, a first device interoperation module 213, and a second device interoperation module 214.

The mobile app. 210 may be installed on the user terminal 20, which is a mobile smart device, and connected to an external smart diet device, that is, a portable device 10, through the smart diet interoperation module 211 to acquire body composition information and activity information from the portable device 10.

Also, the mobile app. 210 may acquire activity measurement information from an external activity measurement device connected through the interoperation module 213 or 214. In this case, the external device includes Fitbit tracker or other activity measurement devices (e.g., Fit guider) which are activity bands developed by Fitbit Inc. and may provide an open API.

According to this embodiment, the user terminal 20 may have an additional component (an interoperation module) for interoperation with an activity measurement device and provide a flexible service through the additional component.

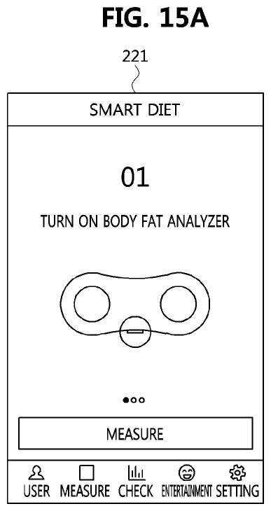

FIG. 12 is a sequence diagram illustrating operating principles of a user terminal that can be employed in the healthcare system of FIG. 1 according to another embodiment.

Referring to FIG. 12, a user terminal 20 of this embodiment may be connected to an SNS platform 50 which provides various social services through a client app. 210A installed thereon.

For social service and personal data management, the client app. 210A may include a personal account login module and an SNS account login module that can be accessed with an SNS platform account. The SNS account login module may include a login module employing an API.

The SNS platform 50 may include a Facebook service providing server, a Twitter service providing server, a Google search service providing server, a Naver portal service providing server, a Nate portal service providing server, a Yahoo portal service providing server, and the like.

A login process of a client app. (hereinafter, “service app.” in brief) 210A in which a login module is installed is as follows by way of example.

First, the user terminal 20 accesses the service app. according to a user input (S101). The service app. 210A performs a login through the SNS platform on the basis of the user input (S102).

As the service app. 210A performs the login, the user terminal 20 logs into the service app. through the SNS platform 50 (S103). Then, the SNS platform 50 may check a qualification (S104).

Subsequently, the user terminal 20 may receive a redirect signal which includes an authentication code and is transferred from the SNS platform 50 to the service app. (S105). The user terminal 20 may access the service app. 210A according to a redirect uniform resource locator (URL) (S106). The service app. 210A may transfer the authentication code and the identity (ID) and the password or secret code of a user or a client to the SNS platform 50 (S107). Then, the service app. 210A may receive an access or valet token from the SNS platform 50 in response (S108). After that, the service app. 210A may provide or output/display information related to the user who has logged into the service app. 210A to the user terminal 20 (S109).

When the above-described mobile app. or client app. (the service app.) is used, it is possible to provide an environment for efficiently planning or developing a community service which induces competition or cooperation. Also, it is possible to provide an activity data interface for checking activity information separately from body composition measurement results. Further, according to implementation, it is possible to advance an app. by additionally installing a foreign language pack or a foreign language support module in order to support various languages or provide an app. of which UI or user experience (UX) has been improved.

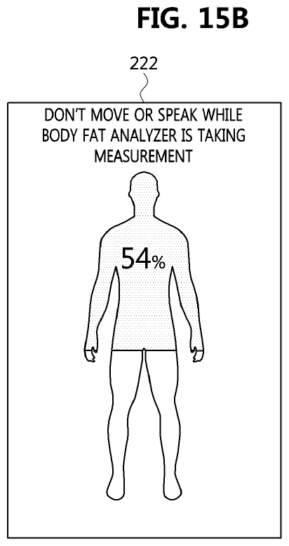

FIG. 13 is a flowchart of a method of providing healthcare information according to another embodiment of the present invention.

Referring to FIG. 13, the method of providing healthcare information according to this embodiment may include a collection step (S110), a generation step (corresponding to S120), and an acquisition step (S130) performed by a cloud-based server.

The collection step may include the operation of collecting healthcare information including body fat measurement results from a portable device for measuring a user's body fat (S110).

The generation step may include the operation of generating group- or region-specific reference data on the basis of the collected body fat measurement results (corresponding to S120).

In other words, in the generation step, the collected data is divided into normal data and exceptional data according to whether the collected data corresponds to a group or region and whether the collected data belongs to an average range, an average value of the exceptional data and the normal data is calculated by applying a preset weight to the exceptional data according to a ratio or distribution (or deviation) of exceptional data to all data of the group or region, and then body fat measurement results customized to a user may be provided by comparing actual body fat measurement data of the user with reference data generated on the basis of the average value.

More specifically, in the generation step, it may be determined first whether a preset group or region corresponds to the previously collected body fat measurement results (S122).

Subsequently, when a preset group or region does not correspond to the collected body fat measurement results, a group or region corresponding to the collected body fat measurement results may be generated (S123).

When a preset group or region corresponds to the collected body fat measurement results or after a group or region is generated, it may be determined whether the collected body fat measurement results are within a preset average range of the group or region (S124).

When it is determined that the collected body fat measurement results are within the average range, the collected information or the collected data may be stored in the corresponding group or region area (S126). Meanwhile, when it is determined that the collected body fat measurement results are not within the average range, an exceptional group of the corresponding group or region may be generated (S127). A preset weight may be applied according to a distribution of previously collected body fat measurement results belonging to the exceptional group or a ratio of the previously collected body fat measurement results to body fat measurement results of the corresponding group or region (S128). Exceptional data to which a weight has been applied may be stored in a separate area of a storage device such as a memory.

After the collected data within the average range is stored in the corresponding group or region area or a weight is applied the collected data of the exceptional group, reference data may be generated on the basis of the collected information (corresponding to first collection data) which is stored to correspond to the group or region and the collected information (corresponding to second collection data) to which the weight has been applied (S129).

The reference data may be an average value of the first collection data and the second collection data. To the second collection data, a preset weight may be applied according to a ratio of the number of pieces of data in the second collection data to the total number of pieces of data in the first collection data and the second collection data. In another implementation manner, a weight preset according to a distribution or deviation based on a difference between a first average value of the first collection data and a level or value of each piece of data in the second collection data may be applied to the second collection data.

The acquisition step may include an operation of acquiring individual body fat measurement result information on the basis of the generated reference data (S130).

Although this embodiment has been described above centering on a body fat measurement function, a method of this embodiment is not limited to a configuration for body fat measurement and may also be applied to an activity measurement function for measuring the amount of activity included in healthcare information in the substantially same way.

According to this embodiment, it is possible to provide information for a user's healthcare, such as obesity management, through accurate body composition measurement.

In other words, body composition measurement employing the healthcare information providing method of this embodiment is essential to obesity management and has very high marketability due to lifestyle characteristics of modern people. Therefore, the body composition measurement may be applied to the disease management field of diabetes, hypertension, and the like.

As described above, according to this embodiment, it is possible to perform a conventional body composition measurement with reliability. As a method for diagnosing obesity, a body mass index (BMI) (kg/m²) obtained by dividing the weight of a person by the square of his or her height is most widely used, and the BMI is an obesity index which has proved correlations with prevalence rates and death rates of obesity-related diseases. However, obesity refers to a state in which body fat is excessively accumulated beyond necessity, and overweight may not mean excessive fat accumulation. Therefore, it is necessary to measure the amount of body fat to accurately diagnose obesity. Also, in the case of evaluating a health risk of a patient with obesity, it is important to measure intra-abdominal visceral fat because whether the patient has abdominal obesity, particularly, the accumulation of visceral fat, is highly correlated with the development of obesity-related diseases even when the patient has the same amount of body fat. Conventional methods for measuring body composition include dual energy X-ray absorptiometry (DXA), underwater weighing, potassium determination, moisture measurement, neutron activation analysis, ultrasonic waves, computer tomography (CT), magnetic resonance imaging (MRI), and the like. However, these methods involve a complex measurement process and complex interpretation and require large facilities. In most cases, these methods are used for the purpose of research rather than a clinical purpose. Therefore, this embodiment may provide a healthcare information providing method employing a portable device (a body composition measuring device), which involves a simple test method, has a low price, is simply and safely carried due to its volume as small as one finger of an adult, and thus can be clinically widely used, using a portable device employing BIA and a cloud-based server connected to the portable device.

Also, it is possible to serve healthcare information about current and past status of a user's body shape or characteristics on the basis of body composition measurement results or activity measurement results, particularly, body composition measurement results, so that the user can easily and conveniently check the healthcare information through a mobile app.

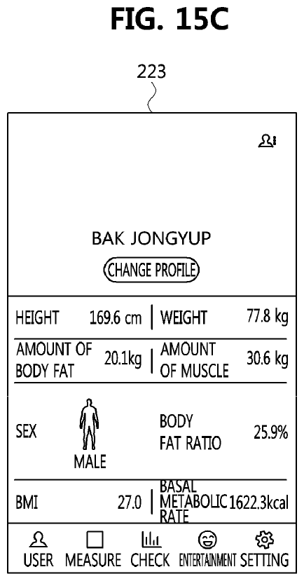

FIG. 14 illustrates operating principles of the healthcare information providing method of FIG. 13.

Referring to FIG. 14, the user terminal 20 displays a UI on a screen 220 thereof by an installed mobile app. or client app. according to a user input.

When the user terminal 20 is used, a user can easily and conveniently measure body fat or the amount of activity and register, edit, and manage healthcare information about the body fat or the amount of activity. According to implementation, the user terminal 20 supports access to an SNS platform and thus can receive a competition or cooperation service through a community.

Also, according to this embodiment, data collected through the user terminal 20 may be stored in a database through a cloud-based server.

The collected data may be stored together with the user's regional information using global positioning system (GPS) information and location information of the mobile app. in addition to a data transmission interface. In this case, the regional information may be used as a classification criterion for the collected data.

Examples of a data interface of a database for classifying collected data on the basis of regional information are shown in [Table 4] and [Table 5].

TABLE 4

| | | | DATA | |
|---|---|---|---|---|
| CMD NAME (Host Request) | VALUE | LEN | Example | Description |
| CMD_GET_VERSION | 0x01 | 0 | NULL | |
| CMD_GET_HBF_VOLT | 0x02 | 0 | NULL | |
| CMD_GET_BATT_VOLT | 0x03 | 0 | NULL | |
| CMD_GET_STAT_VOLT | 0x04 | 0 | NULL | |
| CMD_GET_ALL_VOLT | 0x05 | 0 | NULL | |
| CMD_GET_BATT_PERCENT | 0x06 | 0 | NULL | |
| CMD_SET_START_MEASURE | 0x07 | 7 | 1750d | [0]: height(2B), |
| | | | 700d | [1]: height(2B), |
| | | | 400d 1 | [2]: age(2B), [3]: sec(1B) |
| CMD_SET_ACCEL_COEFF | 0x0A | 24 | 1.0-1.1 | [0]: A0(4B), [1]: A1(4B), |
| | | | 0.4 | [2]: A2(4B), [3]: B0(4B), |
| | | | 0.06 | [4]: B1(4B), [5]: B2(4B) |
| | | | 0.1 | |
| | | | 0.06 | |
| CMD_GET_ACCEL_COEFF | 0x0B | 0 | NULL | Same as above |
| CMD_GET_ACCEL_DATA | 0x0C | 0 | NULL | [0]: buff_count(1B), |
| | | | | [1]: accel_x(2B), |
| | | | | [2]: accel_y(2B), |
| | | | | [3]: accel_z(2B) |
| CMD_SET_PEDOMETER_MODE | 0x10 | 1 | 0x00 | 0: PEDOMETER_XYZ, |
| | | | | 1: PEDOMETER_DISABLE, |
| | | | | 2 = PEDOMETER_ENABLE |
| CMD_GET_PEDOMETER_Mode | 0x11 | 0 | NULL | |
| CMD_SET_STEPCOUNT_CLEAR | 0x12 | 0 | NULL | |
| CMD_GET_STEPCOUNT | 0x13 | 0 | NULL | |

TABLE 5

| Device | | | | |
|---|---|---|---|---|
| Response | | | DATA | |
| CMD | VALUE | LEN | Example | Description |
| 0x80\| | 0x01 | 1 | 0x21 | version = 2.1 |
| Host_CMD | | | | |
| Same as | 0x02 | 2 | 1234d(0x04D2) | hbf_volt, sensing |
| above | | | | voltage = 1.234 V, |
| | | | | transmission |
| | | | | sequence = 0xD2 0x04 |
| Same as | 0x03 | 2 | 3925d(0x0F55) | batt_volt, battery |
| above | | | | voltage = 3.952 V, |
| | | | | transmission |
| | | | | sequence = 0x55 0x0F |
| Same as | 0x04 | 2 | 952d(0x03B8) | stat_volt, STAT |
| above | | | | voltage = 0.952 V, |
| | | | | transmission |
| | | | | sequence = 0xB8 0x03 |
| Same as | 0x05 | 8 | 1234d, 3925d, | [0]: hbf_volt(2B), |
| above | | | 952d, 3300d | [1]: batt_volt(2B), |
| | | | | [2]: stat_volt(2B), |
| | | | | [3]: vcc_volt(2B) |
| Same as | 0x06 | 3 | 1d, 1000d | [0]: charging_status(1B), |
| above | | | | [1]: batt_percent(2B) |
| Same as | 0x07 | 14 | | [0]: charging_status(1B), |
| above | | | | [1]: batt_percent(2B), |
| | | | | [2]: measure_result(1B) |
| | | | | [3]: pbf(2B), |
| | | | | [4]: mbf(2B), |
| | | | | [5]: mesule(2B), |
| | | | | [6]: bmi(2B), |
| | | | | [7]: bmr(2B) |
| Same as | 0x0A | 24 | Return same | [0]: A0(4B), |
| above | | | received data | [1]: A1(4B), |
| | | | | [2]: A2(4B), |
| | | | | [3]: B0(4B), |
| | | | | [4]: B1(4B), |
| | | | | [5]: B2(4B) |
| Same as | 0x0B | 24 | Same as above | Same as above |
| above | | | | |
| Same as | 0x0C | (buff_count*6)+1 | buff_count, | [0]: buff_count(1B), |
| above | | | accel_x, | [1]: accel_x(2B), |
| | | | accel_y, | [2]: accel_y(2B), |
| | | | accel_z | [3]: accel_z(2B) |
| Same as | 0x10 | 1 | | |
| above | | | | |
| Same as | 0x11 | 1 | | |
| above | | | | |
| Same as | 0x12 | 0 | | |
| above | | | | |
| Same as | 0x13 | 4 | step_count(4B) | |
| above | | | | |

When the above-described data interface is used, the mobile app. or client app. (a service app.) can provide a body shape analysis service corresponding to measurement results by providing a body shape analysis service so that a user can intuitively know his or her body fat measurement information. Also, the mobile app. or client app. can collect and classify body composition measurement data by country or region, provide average data on the basis of a classified region, and thereby provide a service for comparatively checking the user's current measurement result values and average values.

Further, when the above-described data interface is used, it is possible to develop content for activity competition among users or with artificial intelligence (AI) provided by a system and provide a service for motivating a user to actively increase the amount of activity.

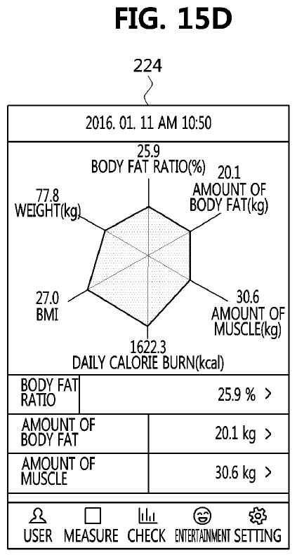

FIGS. 15A to 15D illustrate operating principles of the healthcare information providing method of FIG. 13 according to other embodiments.

As shown in FIG. 15A, a user may display a first screen 221 for healthcare management on a user terminal by selecting a user input through a UI of the user terminal. In the first screen displayed on the user terminal, “smart diet” is shown as the name of a mobile app., and guide words for turning on a body fat analyzer according to a user input and a measurement button for a user input for starting body composition measurement are shown.

When body composition measurement is started, as shown in FIG. 15B, guide words indicating that body fat measurement is underway and a dynamic image or graph of body fat measurement status may be shown in a second screen 222 displayed through a display device of the user terminal or the UI of the mobile app.

Subsequently, as shown in FIG. 15C, a third screen 223 may be displayed on the display device of the user terminal. The third screen 223 may include a link button for accessing a UI for changing the user's name and profile and an area in which information on his or her height, weight, amount of body fat, amount of muscle, sex, body fat ratio, BMI, and basal metabolic rate is shown.

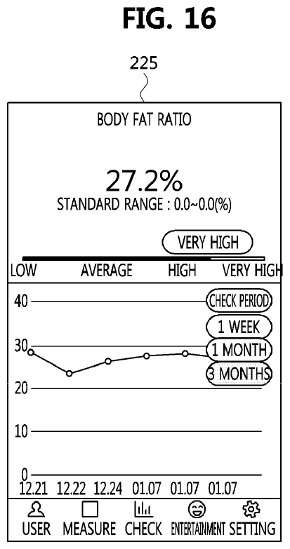

In response to a user input on a check button positioned at the lower end of a fourth screen 224 of a UI, as shown in FIG. 15D, while the user's weight, body fat ratio, amount of body fat, amount of muscle, BMI, and daily calorie burn are displayed as a radar chart, a UI showing link buttons representing that there are details of the body fat ratio, the amount of body fat, and the amount of muscle may be provided as well. Detailed information of the body fat ratio is shown in FIG. 16.

FIG. 16 illustrates operating principles of the healthcare information providing method of FIG. 13 according to another embodiment.

Referring to FIG. 16, a UI of a user terminal of this embodiment may include a fifth screen 225 for displaying detailed information of a body fat ratio. In the fifth screen 225, individual body fat measurement results based on reference data are shown to be “very high.”

Also, as a user selects a check period of three months, the fifth screen 225 may show information on body fat ratios of three months by measurement date in the form of a broken-line graph.

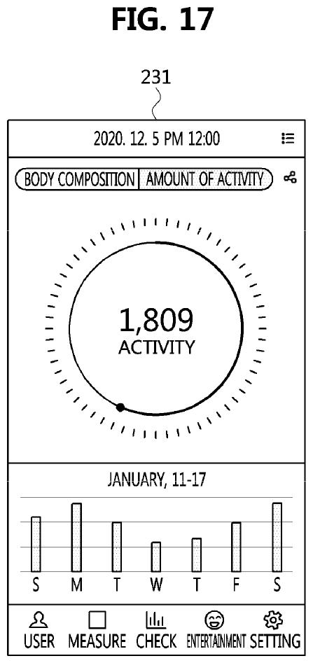

FIG. 17 illustrates operating principles that can be employed in the healthcare information providing method of FIG. 13 according to another embodiment.

Referring to FIG. 17, a UI of a user terminal of this embodiment may include a sixth screen 231 for displaying detailed information on the amount of activity. The number of steps which is the current amount of daily activity may be shown to be “1,809” in the upper middle portion of the sixth screen 231, and the numbers of steps of one last week may be shown by day of the week in the form of a bar graph.

Such a number of steps may be measured and displayed or managed using the sensor and the step count recognition module of the portable device described above.

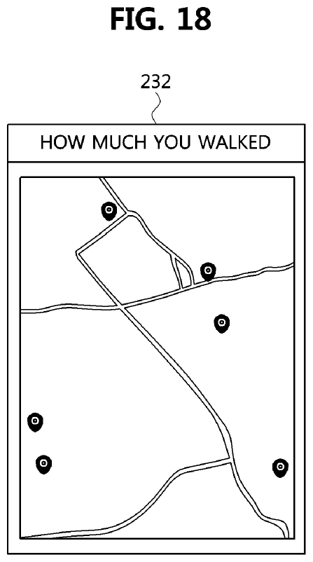

FIG. 18 illustrates operating principles of FIG. 17 according to another embodiment.

Referring to FIG. 18, a UI of a user terminal of this embodiment may include a seventh screen 232 for displaying detailed information on the amount of activity. In the seventh screen 232, information on the number of steps which is the current amount of daily activity may be shown as measurement locations on a map. In this case, a mobile app. of the user terminal may have a GPS module or a location information module installed thereon or interoperate with the module.

A device using the healthcare information providing method according to this embodiment (hereinafter, a “computing device” in brief) may be composed of a control unit including a processor or a microprocessor and a storage unit including a memory. The control unit may be connected to a communication unit through a communication interface.

The processor may include at least one central processing unit (CPU). The CPU may be implemented as a system on chip (SOC) in which a micro-control unit (MCU) and a peripheral device (an integrated circuit for an external expansion device) are disposed together, but the CPU is not limited thereto. A core includes a register for storing instructions to be processed, an arithmetic logical unit (ALU) for comparisons, judgements, and arithmetic operations, a control unit for internally controlling the CPU to interpret and execute instructions, an internal bus, and the like.

Also, the processor may include, but is not limited to, at least one data processor, image processor, or codec. The data processor, image processor, or codec may be configured separately. The processor may further include a peripheral device interface and a memory interface. In this case, the peripheral device interface may be used to connect the processor to an input/output system and several other peripheral devices, and the memory interface may be used to connect the processor and the memory.

To perform a data encryption method, the above-described processor may perform data input, data processing, and data output by executing various software programs. The processor may execute a particular program or software module (an instruction set) stored in the memory and perform several particular functions corresponding to the module. In this embodiment, the processor may cooperate with the cloud-based server by executing software modules stored in the memory 20m and thereby output individual body fat measurement results based on group- or region-specific collected information or healthcare information including the individual body fat measurement results through the user terminal or display the output individual body fat measurement results or the healthcare information on a display device of the user terminal.

The storage unit may include one or more fast random access memories and/or non-volatile memories, such as magnetic disc storage devices, one or more optical storage devices, and/or a flash memory. The storage unit may store an operating system, software, a program, an instruction set, or a combination thereof.

The operating system includes an embedded operating system such as MS WINDOWS, LINUX, Darwin, RTXC, UNIX, OS X, iOS, MAC OS, VxWorks, Google OS, Android, Bada (Samsung OS), Plan9, etc. and may have several components for controlling system operations of the user terminal including a mobile device and the like. The operating system may have, but is not limited to, the function of performing communication between various pieces of hardware (devices) and software components (modules).

The software components may include an operating system module, a communication module, a graphic module, a UI module, a moving picture experts group (MPEG) module, a camera module, one or more application modules, and the like. A module is a set of instructions and may be referred to as an instruction set or a program.

The communication interface supports one or more communication protocols so that the user terminal can be connected to a server system, a file server, a database server, or other network devices via a network. The communication interface may include one or more wireless communication sub-systems. The wireless communication sub-system may include a radio frequency receiver and transceiver and/or an optical (e.g., infrared) receiver or transceiver.

The network may include, for example, a global system for mobile communication (GSM) network, an enhanced data GSM environment (EDGE) network, a code division multiple access (CDMA) network, a W-code division multiple access (W-CDMA) network, a long term evolution (LTE) network, an LTE-advanced (LTE-A) network, an orthogonal frequency division multiple access (OFDMA) network, a worldwide interoperability for microwave access (WiMAX) network, a wireless fidelity (Wi-Fi) network, a Bluetooth network, and the like.

Meanwhile, in this embodiment, components used to implement the healthcare information providing method may be, but are not limited to, functional blocks or modules installed in a user terminal or a computer device. The above-described components may be stored in a computer-readable medium (recording medium) in a software form for implementing a series of functions (the healthcare information providing method) performed by the components or may be transmitted to a remote site in a carrier form and operate in various computer devices. The computer-readable medium may include a medium combined with a plurality of computer devices or a cloud system connected via a network. A program, source code, etc. enabling a user terminal or a cloud-based server to implement the healthcare information providing method may be stored in the computer-readable medium.

In other words, the computer-readable medium may be implemented to include program instructions, data files, data structures, etc. separately or in combination. The program recorded in the computer-readable medium may be specially designed and configured for the present invention or may be known and available to those of ordinary skill in the computer software field.

The computer-readable medium may include a hardware device specially configured to store and execute program instructions, such as a read only memory (ROM), a random access memory (RAM), and a flash memory. The program instructions may include not only machine language codes created by a compiler but also high-level language codes that can be executed by a computer using an interpreter or the like. The hardware device may be configured to operate as at least one software module in order to perform the healthcare information providing method of this embodiment, and vice versa.

When the method of providing healthcare information using a cloud-based portable device for measuring body fat and the device using the method according to the above-described embodiments of the present invention are used, it is possible to provide a portable device which is as small as one finger of an adult and capable of measuring a user's body composition and amount of activity and provide a service app. for providing the function of cooperating with a social network of the user on the basis of the amount of activity and/or information related to the body composition or a user terminal on which the service app. is installed.

A user can measure his or her body composition and/or the amount of activity regardless of place and time while carrying a small-sized portable device, and can enhance a user community by frequently providing relevant information to a cloud-based server or a social network of the user.

Individual body fat measurement results are regenerated by reprocessing body fat measurement results collected from a user's portable device or a user terminal with reference data including an average value based on a preset group or region such that reliable body fat measurement results can be provided to each user. Also, since a cloud-based server for providing such a healthcare information service is used, it is advantageous to plan or develop a community service for inducing competition and/or cooperation.

Further, it is possible to provide a healthcare app. in which body fat measurement results, activity information, healthcare information sharing, community services, etc. are integrated with a UI or UX. Here, the healthcare information may include step count recognition, collected health data, and the like.

Although the present invention has been described above with reference to exemplary embodiments, those of ordinary skill in the art should understand that the present invention can be altered and modified in various ways within the spirit and scope of the present invention set forth in the following claims.

1. A method of providing healthcare information from a cloud-based server using a cloud-based portable device for measuring body fat, the method comprising:
collecting body fat measurement results from portable devices for measuring a user's body fat;
generating group- or region-specific reference data on the basis of the collected body fat measurement results; and
acquiring individual body fat measurement result information on the basis of the generated reference data.

2. The method of claim 1, wherein the generating of the reference data comprises:
determining whether a preset group or region corresponds to the collected body fat measurement results; and
generating a group or region corresponding to the collected body fat measurement results when any preset group or region does not correspond to the collected body fat measurement results.

3. The method of claim 2, wherein the generating of the reference data further comprises, after the generating of the group or region:
determining whether the collected body fat measurement results are within a preset average range of the group or region;
generating an exceptional group of the group or region when the collected body fat measurement results are not within the preset average range; and
applying a weight preset according to a distribution of pre-collected body fat measurement results belonging to the exceptional group or a ratio of the pre-collected body fat measurement results to the body fat measurement results of the group or region.

4. The method of claim 3, wherein the generating of the reference data further comprises, after the applying of the weight, generating reference data on the basis of collected information stored in connection with the group or region and stored collected information to which the weight has been applied.

5. A user terminal which is connected to a cloud-based server via a network and has a mobile application for outputting individual body fat measurement results, the user terminal comprising:
a memory configured to store a program; and
a processor configured to be connected to the memory and execute the program,
wherein the program includes a body fat analysis module, a step count recognition module, and a health data collection module, and
due to the program, the processor acquires body fat measurement results from a portable device for measuring a user's body fat, transmits the acquired body fat measurement results to the cloud-based server, and acquires individual body fat measurement results based on reference data, which is generated on the basis of body fat measurement results collected according to groups or regions, from the cloud-based server.

6. The user terminal of claim 5, wherein the reference data is generated on the basis of an average of first collected information within an average range preset according to the groups or regions and second collected information which is not within the average range and to which a weight has been applied.

7. A portable device which is connected to a user terminal via a wireless network and measures a user's body fat, the portable device comprising:
a first pair of terminals with which a thumb and one of other fingers of the user's left hand each come in contact;
a second pair of terminals with which a thumb and one of other fingers of the user's right hand each come in contact;
at least one signal detection circuit configured to be connected to the first pair of terminals and the second pair of terminals and measure a signal related to the user's body composition or healthcare;
a controller configured to be connected to the signal detection circuit;
a sensor configured to be connected to the controller and detect vibration or acceleration; and
a communication unit configured to be connected to the controller and connected to the user terminal via a communication network,
wherein the controller transmits the signal or information corresponding to the signal to a cloud-based server through the user terminal, and
the cloud-based server classifies collected body fat measurement results by preset group or region, acquires individual body fat measurement results on the basis of reference data generated on the basis of a group- or region-specific average, and provides the individual body fat measurement results to the user terminal.

8. The portable device of claim 7, wherein the reference data is generated on the basis of an average of first collected information within an average range preset according to groups or regions and second collected information which is not within the average range and to which a weight has been applied.
